(19) United States
(12) Patent Application Publication
(10) Pub. No.: US 2021/0178061 A1
(43) Pub. Date: Jun. 17, 2021

(54) MEDICAL INFUSION SYSTEM

(71) Applicant: B. Braun Melsungen AG, Melsungen (DE)

(72) Inventor: Andreas Katerkamp, Melsungen (DE)

(21) Appl. No.: 17/270,126

(22) PCT Filed: Jul. 1, 2019
(86) PCT No.: PCT/EP2019/067624

§ 371 Date: Feb. 22, 2021

(30) Foreign Application Priority Data
Aug. 23, 2018 (DE) 10 2018 214 249.1

(51) Int. Cl. A61M 5/168; A61M 39/20; A61M 5/14; A61M 39/24; A61M 5/142

(52) U.S. Cl. CPC A61M 5/16854; A61M 39/20; A61M 5/1411; A61M 39/24; A61M 2205/7536; A61M 2205/3331; A61M 2205/581; A61M 2205/58; A61M 5/142

(57) ABSTRACT

A medical infusion system includes a hose line with a distal end region for connection to an infusion reservoir and a proximal end region that has a patient connection port. The patient connection port has a closure device with an air-permeable and liquid-tight design. The system can be used in the medical sector for intravenous medication.

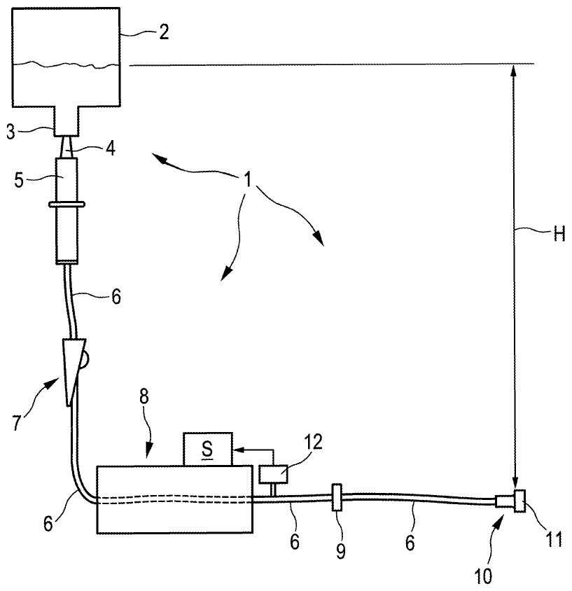

### CROSS-REFERENCE TO RELATED APPLICATION(S)

This application is the United States national phase entry of International Application No. PCT/EP2019/067624, filed Jul. 1, 2019, and claims the benefit of priority of German Application No. 10 2018 214 249.1, filed Aug. 23, 2018. The contents of International Application No. PCT/EP2019/067624 and German Application No. 10 2018 214 249.1 are incorporated by reference herein in their entireties.

### FIELD

The invention relates to a medical infusion system having a hose line with a distal end region for connection to an infusion reservoir and with a proximal end region that has a patient connection port.

### BACKGROUND

U.S. Pat. No. 5,954,485 A discloses a free-flow protection device for an infusion pump of a medical infusion system, wherein the free-flow protection device prevents a free flow of infusion liquid to a patient connection port. The device is part of a medical infusion system in which infusion liquid from an infusion reservoir flows through a hose line to a patient connection port. During the operation of the infusion system, the patient connection port is connected to a patient-side catheter. If, during the operation of the infusion system, the infusion reservoir is suspended above the patient connection port and the patient connection port is connected to the catheter of the patient, then, without additional measures, infusion liquid flows freely into a vein of the patient until a blood pressure of the patient and the hydrostatic pressure of the infusion system have equalized each other. A free flow of this kind can be disadvantageous, since it can lead to overmedication of the patient. Moreover, air bubbles in the hose line, which enter the blood stream of the patient, can have unwanted consequences. U.S. Pat. No. 5,954,485 discloses a device which prevents such a free flow of infusion liquid. The device comprises a clamp region which, depending on the position of a lever, closes or frees the hose line. The device is assigned to an infusion pump which controls the flow of the infusion liquid through the hose line.

### SUMMARY

The object of the invention is to make available a medical infusion system which is of the type mentioned at the outset and which allows a first filling of the infusion system to take place in a manner that is safe and free of contamination.

For the medical infusion system, this object is achieved by the fact that the patient connection port is provided with a closure device, which is of an air-permeable and liquid-tight design. The solution according to the invention permits a first filling of the hose line and a connection of the patient connection port to a patient access, in particular a patient-side catheter, without any possibility of contamination of the user or of the patient in the region of the patient connection port. The air permeability of the closure device ensures that air bubbles arising in the hose line during the first filling are able to escape in the region of the patient connection port. The liquid-tight design at the same time ensures that no infusion liquid can escape in the region of the patient connection port during the first filling. The closure device is preferably designed in such a way that it is only effective until such time as the infusion system is attached ready for operation at the patient side, in order not to impede the infusion of infusion liquid, which is desired after the ready-for-operation attachment, to the patient access, in particular via a catheter into a vein of the patient. The terms “distal end region” and “proximal end region” are in relation to a patient, i.e. the patient connection port of the infusion system is near the patient, i.e. proximal, and the end region for connection to the infusion reservoir is remote from the patient and thus distal with respect to the hose line.

In one embodiment of the invention, the closure device has a closure cap, which is held in a pressure-tight manner on the patient connection port. This ensures that the closure cap is not accidentally detached from the patient connection port by a delivery pressure generated by an infusion pump.

In a further embodiment of the invention, the patient connection port or the closure cap has a closure membrane, which closes the patient connection port in such a way that the latter is permeable to air and repels infusion solution. Preferably, the closure cap with the closure membrane remains on the patient connection port during a connection of the patient connection port to the patient access. Alternatively, the closure membrane is connected to the patient connection port in such a way that, even after the removal of the closure cap, the closure membrane remains in the region of a corresponding through-opening of the patient connection port. The closure membrane is advantageously pierced through automatically, as soon as the patient connection port is connected to the patient access.

In a further embodiment of the invention, the distal end region has a drip chamber with a piercing mandrel. The piercing mandrel is provided to connect the drip chamber to a closure stopper of an infusion reservoir.

In a further embodiment of the invention, the hose line is assigned a throughflow control device, in particular in the form of a roller clamp. The hose line, the drip chamber, the roller clamp and the patient connection port form a transfer system for the medical infusion system, in order to convey infusion liquid from the infusion reservoir to a patient access.

In a further embodiment of the invention, the hose line is assigned a check valve with an opening pressure greater than a hydrostatic pressure of the infusion system, which check valve lies in the proximal end region of the hose line in the assembled operating state. The hydrostatic pressure of the infusion system arises from the fact that the infusion reservoir filled with infusion liquid is positioned at a height above a patient access, preferably about 1 m above the patient access. The check valve serves as a free-flow protection, since it keeps the hose line closed as long as only the hydrostatic pressure of the infusion system is effective inside the hose line. If the infusion reservoir is positioned about 1 m above the patient access, the hydrostatic pressure inside the hose line is about 100 mbar. Accordingly, an opening pressure of the check valve is advantageously about 150 mbar.

In a further embodiment of the invention, the hose line is assigned an infusion pump which, during operation, exerts a pressure, greater than the opening pressure of the check valve, on an infusion liquid that is to be delivered. Preferably, during operation, the infusion pump applies a delivery pressure of 200 mbar to the infusion liquid that is to be delivered, such that the operation of the infusion pump opens the check valve and permits the flow of the infusion liquid through the hose line. The check valve is advantageously assigned to the hose line in the region between the infusion pump and the patient connection port.

In a further embodiment of the invention, the closure membrane opens at a water breakthrough pressure that is greater than a delivery pressure of the infusion pump. This ensures that, during a first filling of the hose line, the patient connection port remains closed even when the infusion pump has started its operation. This permits a complete first filling of the hose line in a manner free of air bubbles, such that, when the patient connection port is connected to the patient access, infusion liquid can be fed into a vein of the patient without the patient connection port or the patient access being contaminated. At a delivery pressure of the infusion pump of about 200 mbar, a water breakthrough pressure of the closure membrane is at least 250 mbar.

In a further embodiment of the invention, the hose line, at the output side of the infusion pump, is assigned a pressure sensor which detects a pressure increase that exceeds the delivery pressure of the infusion pump. The pressure sensor detects a pressure increase exceeding the delivery pressure which arises when a first filling of the hose line is concluded and the infusion pump continues to deliver infusion liquid in the direction of the patient connection port.

In a further embodiment of the invention, the pressure sensor is attached to a signal device in such a way that the signal device emits a signal as soon as a pressure increase that exceeds the delivery pressure is detected. The signal device preferably emits an optical and/or acoustic signal. The signal serves to inform a user that the first filling process is concluded, and that a drive of the infusion pump can be switched off.

In a further embodiment of the invention, the pressure sensor is attached to a control device of the infusion pump in such a way that, when a pressure increase that exceeds the delivery pressure is detected, a drive of the infusion pump is switched off and/or the signal device is actuated to generate an optical and/or acoustic signal. In this way, the infusion pump is switched off in an automated manner as soon as the first filling process is concluded. Alternatively or in addition, when a pressure of 200 mbar is detected, the pressure sensor emits a corresponding signal which the control device processes and/or which is optically and/or acoustically discernible to a user.

Further advantages and features of the invention will become clear from the following description of a preferred exemplary embodiment of the invention, which is shown in the drawing.

### BRIEF DESCRIPTION OF THE DRAWING

The single drawing is a schematic representation of an embodiment of a medical infusion system according to the invention.

### DETAILED DESCRIPTION

An infusion system 1 has a transfer system between an infusion reservoir 2 and a patient access (not shown). The medical infusion system 1 serves to convey infusion liquid from the infusion reservoir 2 to the patient access, in order to administer a medication or a nutrient solution to a patient. The patient access provided is in particular an intravenous patient access to which the infusion system 1 is connectable.

The medical infusion system 1 has a flexible hose line 6, which is advantageously transparent. The hose line 6 is firmly connected, at a distal end region, to a drip chamber 5, which is provided distally with a piercing mandrel 4. When the infusion system 1 is in an assembled state ready for operation, the piercing mandrel 4 has been inserted with force-fit engagement into an elastically resilient closure stopper 3 in the region of an underside of the infusion reservoir 2. The piercing mandrel 4 has at least one channel which protrudes into the interior of the infusion reservoir 2 and through which infusion liquid from the infusion reservoir 2 is conveyed into the drip chamber 5 and from there into the hose line 6. In the assembled state ready for operation, the infusion reservoir 2 is suspended in a stationary position, in particular on a stand. The stand is advantageously provided with rollers, so as to be able to move the infusion reservoir 2, including the infusion system 1, on a floor surface.

In the direction of flow of the infusion system 1, the drip chamber 5 is followed by a roller clamp 7, which is provided for manually controlling a flow of the infusion liquid through the hose line 6. The infusion system 1 is additionally assigned a check valve 9 (described in more detail below), and, at a proximal end region of the hose line 6, a patient connection port 10. The patient connection port 10 is closed by a closure cap 11.

In the assembled state ready for operation, the hose line 6 is attached to an infusion pump 8, which is provided with a drive (not shown in detail) so as to act mechanically on a jacket of the hose line 6 and thereby convey the infusion liquid through the hose line 6. For this purpose, the hose line 6 is placed, in a manner basically known per se, into a corresponding receiving region of the infusion pump 8 and fixed in the receiving region.

Finally, at the output side of the infusion pump 8, the hose line 6 is assigned a pressure sensor 12, which is attached to a control device S (described in more detail below).

The patient connection port 10 is provided, in a manner not shown in detail, with a closure membrane which closes the patient connection port such that the latter is permeable to air and repels infusion solution, i.e. is liquid-tight. The closure membrane is assigned either directly to a through-opening of the patient connection port 10 or to the closure cap 11. In both cases, the closure membrane serves to close the proximal end of the infusion system 1, and hence the hose line 6, in a liquid-tight manner but at the same time so as to allow air to pass through. If the closure membrane is assigned to the closure cap 11, the closure cap preferably remains on the patient connection port 10 upon connection of the patient connection port 10 to the patient access, in order to automatically achieve separation of the closure membrane upon connection without infusion liquid escaping at a connection site between patient connection port 10 and patient access. The patient connection port 10 and the patient access are preferably provided with mutually complementary Luer lock connectors. If the closure membrane is assigned directly to the patient connection port 10, the closure cap 11 can be removed from the patient connection port 10 without infusion liquid being able to escape from the patient connection port 10. This is because the closure membrane continues to close the patient connection port 10 until the patient connection port 10 is firmly connected to the patient access by means of a corresponding Luer lock connection. During the connection, the closure membrane is advantageously pierced through or severed in some other way, as a result of which a flow of the infusion liquid is enabled. The air permeability of the closure membrane ensures that, when the infusion system 1 is filled for the first time, air bubbles can pass through the hose line 6 to the patient connection port 10 and can escape at the patient connection port 10, such that a first filling with infusion liquid is permitted in a manner free of air bubbles.

To set the infusion system 1 in operation, the piercing mandrel 4 is introduced into the closure stopper 3 of the infusion reservoir 2. The infusion reservoir 2 is already suspended in a stationary position at a height H, which is preferably about 1 m above the patient access. The piercing mandrel 4 is pressed from underneath into the closure stopper 3, until infusion liquid is able to flow through the channel of the piercing mandrel 4 into the drip chamber 5. The roller clamp 7 is opened. The hose line 6 is mounted in the region of the infusion pump 8 provided with an electric drive.

The check valve 9 has an opening pressure that is greater than a hydrostatic pressure resulting from the difference in height between the suspended infusion reservoir 2 and the patient access, i.e. the patient connection port 10. Therefore, as long as the infusion pump 8 is not activated, the check valve 9 closes the hose line 6 in a region between the infusion pump 8 and the patient connection port 10. The check valve 9 defines a free-flow protection device. However, the opening pressure of the check valve 9 is lower than a delivery pressure of the infusion pump 8 as soon as the infusion pump 8 is activated. Accordingly, after activation of the infusion pump 8, the check valve 9 opens on account of the delivery pressure for the infusion liquid then being increased in the hose line 6, such that the infusion liquid is delivered as far as the patient connection port 10. The patient connection port 10 is closed by the closure membrane. Any air bubbles are also conveyed in the direction of the patient connection port 10 together with the infusion liquid. On account of the air permeability of the closure membrane, the corresponding air can escape into the environment in the region of the patient connection port 10. At the same time, the closure membrane is designed to repel infusion solution, such that infusion solution builds up in the region of the closure membrane of the patient connection port 10. As infusion liquid is further delivered by the operation of the infusion pump 8, the pressure in the hose line 6 thus necessarily increases. The corresponding increase in pressure is monitored by the pressure sensor 12, which is provided at the output side of the infusion pump 8. As soon as the pressure inside the hose line increases above the normal delivery pressure of the infusion pump 8, the pressure sensor 12 emits a signal to the control device S, which then actuates an acoustic and/or optical signal generator (not shown) and/or switches off the drive of the infusion pump 8. If only the acoustic and/or optical signal generator is actuated, the user, in particular the hospital personnel, has to switch off the drive of the infusion pump 8 depending on the detection of the corresponding signal. If the control device S actuates the drive of the infusion pump 8 directly, the drive of the infusion pump 8 can switch off automatically when a corresponding pressure increase is detected.

If the height difference H is 1 m, the hydrostatic pressure inside the infusion system 1 is 100 mbar. The check valve 9 has an opening pressure of about 150 mbar. A delivery pressure that is generated by activation of the infusion pump 8 is preferably more than 150 mbar and advantageously a maximum of 200 mbar. The pressure sensor 12 is advantageously designed in such a way that, at a pressure of more than 200 mbar, it sends a corresponding signal to the control device S. At the values stated above, a water breakthrough pressure of the closure membrane is then advantageously at least 250 mbar.

It is thus ensured that a first filling of the infusion system 1 takes place safely and in a manner free of air bubbles. The patient connection port 10 can now be connected to the patient access, as a result of which the closure membrane opens and a flow of the infusion liquid to the patient access is enabled.

1. A medical infusion system having a hose line with a distal end region for connection to an infusion reservoir and with a proximal end region that has a patient connection port, wherein the patient connection port is provided with a closure device, which is of an air-permeable and liquid-tight design.

2. The medical infusion system as claimed in claim 1, wherein the closure device has a closure cap, which is held in a pressure-tight manner on the patient connection port.

3. The medical infusion system as claimed in claim 2, wherein the patient connection port or the closure cap has a closure membrane, which closes the patient connection port in such a way that the latter is permeable to air and repels infusion solution.

4. The medical infusion system as claimed in claim 1, wherein the distal end region has a drip chamber with a piercing mandrel.

5. The medical infusion system as claimed in claim 1, wherein the hose line is assigned a throughflow control device, in particular in the form of a roller clamp.

6. The medical infusion system as claimed in claim 1, wherein the hose line is assigned a check valve with an opening pressure greater than a hydrostatic pressure of the infusion system, which check valve lies in the proximal end region of the hose line in the assembled operating state of the infusion system.

7. The medical infusion system as claimed in claim 6, wherein the hose line is assigned an infusion pump which, during operation, exerts a pressure, greater than the opening pressure of the check valve, on an infusion liquid that is to be delivered.

8. The medical infusion system as claimed in claim 7, wherein the closure membrane opens at a water breakthrough pressure that is greater than a delivery pressure of the infusion pump.

9. The medical infusion system as claimed in claim 8, wherein the hose line, at the output side of the infusion pump, is assigned a pressure sensor which detects a pressure increase that exceeds the delivery pressure of the infusion pump.

10. The medical infusion system as claimed in claim 9, wherein the pressure sensor is attached to a signal device in such a way that the signal device emits a signal as soon as a pressure increase that exceeds the delivery pressure is detected.

11. The medical infusion system as claimed in claim 10, wherein the signal device is configured to emit an optical and/or acoustic signal.

12. The medical infusion system as claimed in claim 11, wherein the pressure sensor is attached to a control device of the infusion pump in such a way that, when a pressure increase exceeding the delivery pressure is detected, a drive of the infusion pump is switched off and/or the signal device is actuated to generate the optical and/or acoustic signal.
